(19) United States
(12) Patent Application Publication
(10) Pub. No.: US 2009/0137827 A1
(43) Pub. Date: May 28, 2009

(54) SILICON-TITANIUM MIXED OXIDE-CONTAINING DISPERSION FOR THE PRODUCTION OF TITANIUM-CONTAINING ZEOLITES

(75) Inventors: Wolfgang Lortz, Waechtersbach (DE); Kai Schumacher, Hofheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: EVONIK DEGUSSA GMBH, ESSEN (DE)

(21) Appl. No.: 12/294,371

(22) PCT Filed: Mar. 12, 2007
(86) PCT No.: PCT/EP07/52278

§ 371 Date: Sep. 24, 2008

(30) Foreign Application Priority Data
Apr. 15, 2006 (DE) 10 2006 017 700.2

(51) Int. Cl. C07D 301/19; B01J 29/04; B01J 29/072

(52) U.S. Cl. 549/529; 502/60; 502/74; 502/62

(57) ABSTRACT

Dispersion containing pyrogenic silicon-titanium mixed oxide powders with a silicon dioxide content of 75 to 99.99 wt.-% and a titanium dioxide content of 0.01 to 25 wt. %, water and a basic, quaternary ammonium compound, wherein the mean aggregate diameter of the particles of the silicon-titanium mixed oxide powder in the dispersion is 200 nm at most. Process for the production of a titanium-containing zeolite with the use of the dispersion.

The invention relates to a silicon-titanium mixed oxide powder-containing dispersion for the production of titanium-containing zeolites.

From EP-A-814058, the use of silicon-titanium mixed oxide powders for the production of titanium-containing zeolites is known. Titanium-containing zeolites are efficient catalysts for the oxidation of olefins with hydrogen peroxide. They are obtained by hydrothermal synthesis starting from silicon-titanium mixed oxide powders in the presence of a template. In EP-A-814058, it is disclosed that pyrogenic silicon-titanium mixed oxides with a silicon dioxide content of 75 and 99.9 wt.-% can be used for this. Particularly advantageous is a composition which contains from 90 to 99.5 wt.-% silicon dioxide and 0.5 to 5 wt. % titanium dioxide. As templates, amines, ammonium compounds or alkali (alkaline earth) metal hydroxides can be used.

A disadvantage of the process disclosed in EP-A-814058 is that it leads to products whose catalytic activity is often not reproducible and is often not adequate.

The purpose of the present invention was therefore to provide a silicon-titanium mixed oxide in a form which makes it possible to produce titanium-containing zeolites of high catalytic activity.

The object of the invention is a dispersion containing pyrogenic silicon-titanium mixed oxide powders with a silicon dioxide content of 75 to 99.99 wt. % and a titanium dioxide content of 0.01 to 25 wt. %, water and a basic, quaternary ammonium compound, wherein the mean aggregate diameter of the particles of the silicon-titanium mixed oxide powder in the dispersion is 200 nm at most.

It was found that on use of this dispersion, which contains particles of this fineness, the reaction time which is needed for the production of titanium-containing zeolites is markedly reduced. Preferably, the mean aggregate diameter is less than 100 nm.

Pyrogenic should be understood to refer to metal mixed oxide particles obtained by flame oxidation and/or flame hydrolysis. During this, oxidisable and/or hydrolysable starting materials are oxidised or hydrolysed as a rule in a hydrogen-oxygen flame. The metal mixed oxide particles according to the invention are as pore-free as possible and have free hydroxyl groups on the surface. They are present in the form of aggregated primary particles.

The BET surface area of the pyrogenic silicon-titanium mixed oxide powders is not limited. It has however been found advantageous if the BET surface area lies in a range from 20 to 400 m²/g and in particular from 50 to 300 m²/g. The use of a silicon-titanium mixed oxide powder of high BET surface area in combination with a small mean aggregate diameter in the dispersion is particularly advantageous for the production of titanium-containing zeolites.

It has further been found advantageous if the dispersion contains a pyrogenic silicon-titanium mixed oxide powder, wherein the proportions of Na, K, Fe, Co, Ni, Al, Ca and Zn are each less than 50 ppm, in particular less than 25 ppm. Such a dispersion leads to titanium-containing zeolites with high catalytic activity.

The dispersion according to the invention also contains a basic, quaternary ammonium compound. These are preferably tetraalkylammonium hydroxides such as for example tetraethylammonium hydroxide, tetra-n-propylammonium hydroxide and/or tetra-n-butylammonium hydroxide. Basic, quaternary ammonium compounds serve as templates which determine the crystal structure through incorporation into the crystal lattice. Tetra-n-propylammonium hydroxide is preferably used for the production of titanium silicalite-1 (MFI structure), tetra-n-butylammonium hydroxide for the production of titanium silicalite-2 (MEL structure) and tetraethylammonium hydroxide for the production of titanium β-zeolites (BEA crystal structure).

The ratio water/silicon-titanium mixed oxide powder is preferably 10≦mol water/ mol silicon-titanium mixed oxide ≦20. The range 12≦mol water/mol silicon-titanium mixed oxide≦17 is particularly preferred.

The content of quaternary, basic ammonium compound in the dispersion according to the invention is not limited. If the dispersion is to be stored for a prolonged period, it can be advantageous to add to it only a part of the quantity of the dispersion necessary for the production of a titanium-containing zeolite. Preferably, the quaternary, basic ammonium compound can be added in a quantity such that a pH value of 9 to 11, in particular 9.5 to 10.5, results. In this pH range, the dispersion displays good stability.

If for example the dispersion is to be used directly after its production for the production of a titanium-containing zeolite, the dispersion can already contain the whole quantity of quaternary, basic ammonium compound. Preferably then 0.12≦mol ammonium compound/mol silicon-titanium mixed oxide <0.20, and 0.13≦mol ammonium compound/mol silicon-titanium mixed oxide≦0.17 is particularly preferred.

A further object of the invention is a process for the production of the dispersion comprising the steps:
water, which, in the event that silicon-titanium mixed oxide powder incorporated later leads to a pH value of the aqueous phase of <2 or >4, is adjusted to pH values of 2 to 4 by addition of acids or bases, is circulated from a holding tank through a rotor/stator machine, and
via a filling device, silicon-titanium mixed oxide powder is introduced continuously or discontinuously into the shear zone between the grooves of the rotor teeth and the stator grooves, with the rotor/stator machine running, in a quantity such that a predispersion with a solids content of 20 to 40 wt.-% results, and
after all the silicon-titanium mixed oxide powder has been added, the filling device is closed, and shearing continued such that the shear rate lies in the range between 10000 to 40000 sec⁻¹, and
then, while maintaining the dispersion conditions, a basic, quaternary ammonium compound is added, and optionally water, before the addition of the ammonium compound.

A further object of the invention is a process for the production of a titanium-containing zeolite, wherein the dispersion according to the invention, optionally with further addition of the basic, quaternary ammonium compound, is processed at a temperature of 150 to 220° C. for a period of less than 12 hours. The crystals obtained are separated by filtration, centrifugation or decantation and washed with a suitable washing liquid, preferably water.

The crystals are then dried as required, and calcined at a temperature between 400° C. and 1000° C., preferably between 500° C. and 750° C., in order to remove the template.

A further object of the invention is a titanium-containing zeolite, which is obtainable by the process according to the invention.

The titanium-containing zeolite is obtained in powder form. For its use as an oxidation catalyst, as required it is converted into a form suitable for the use, e.g. into micropellets, spheres, tablets, solid cylinders, hollow cylinders or honeycombs by known methods for the shaping of catalysts in powder form, such as for example pelleting, spray-drying, spray pelleting or extrusion.

The titanium-containing zeolite according to the invention can be used as a catalyst in oxidation reactions with hydrogen peroxide. In particular, it can be used as a catalyst in the epoxidation of olefins by means of aqueous hydrogen peroxide in a solvent miscible with water.

### EXAMPLES

### Example 1

### Production of a Silicon-Titanium Mixed Oxide Powder

5.15 kg/hr of silicon tetrachloride and 0.15 kg/hr of titanium tetrachloride are vaporised. The vapours are transferred into a mixing chamber by means of 15 Nm³/hr of nitrogen as carrier gas. Separately from this, 2 Nm³/hr of hydrogen and 8 Nm³/hr of primary air are introduced into the mixing chamber. In a central pipe, the reaction mixture is fed into a burner and ignited. Here the flame burns in a water-cooled flame tube. In addition, 15 Nm³/hr of secondary air is introduced into the reaction space. The powder produced is separated in a filter connected downstream and then treated with a counter-current of hydrogen at 520° C.

The powder displays the following values:
Silicon dioxide 96.6 wt. %
Titanium dioxide 3.4 wt. %
BET surface area 80 m2/g

### Example 2

### Production of a Dispersion (According to Invention)

32.5 kg of deionised water are first placed in a 100 l stainless steel batch vessel. Next, 17.5 kg of the silicon-titanium mixed oxide powder from Example 5 are inducted under shear conditions using the suction tube of the Ystral Conti-TDS 4 (Stator groove: 6 mm ring and 1 mm ring, rotor/stator gap ca. 1 mm). After completion of the induction, the inlet nozzle is closed and the 35 weight percent predispersion is sheared for a further 10 mins at 3000 rpm. Undesired warming of the dispersion due to the high energy input is counteracted with a heat exchanger and the temperature rise limited to max. 40° C. Owing to the acidic nature of the pyrogenically produced silicon-titanium mixed oxide powder, the pH value of the dispersion is ca. 3.6.

Next, 28.6 kg of deionised water are added, and a pH value of 10.0 is rapidly established under intensive shearing and thorough mixing with 1.0 kg of tetra-n-propylammonium hydroxide solution (40 wt.-% in water).

The dispersion displays the following values:
Water/silicon-titanium mixed oxide 11.7
Mean aggregate diameter 94 nm (determined on Horiba LA 910)

### Example 3

### Production of a Dispersion (Comparison)

1 g of tetra-n-propylammonium hydroxide solution (40 wt.-% in water) are added to 17.5 g of the silicon-titanium mixed oxide powder from Example 1 in 61.1 ml of water under dispersing conditions by means of a dissolver, and dispersed for 30 mins. The dispersion obtained has a markedly higher viscosity in comparison to Example 3. Coarse aggregates can clearly be discerned as well as finer ones.

The dispersion displays the following values:
Water/silicon-titanium mixed oxide 13.2
Tetrapropylammonium hydroxide/silicon-titanium mixed oxide 0.14
Mean aggregate diameter 256 nm

### Example 4

### Production of a Titanium-Containing Zeolite (According to Invention)

505 g of the dispersion from Example 2 are first placed in a polyethylene beaker, 46.7 g of deionised water and 130.6 g of a tetra-n-propylammonium hydroxide solution (40 wt.-% in water) are added and firstly aged for four hours at 80° C. with stirring and then crystallised for 10 hours at 180° C. in an autoclave. The solid obtained is separated from the mother liquor by centrifugation, washed with 3×250 ml portions of deionised water, dried at 90° C. and calcined in an atmosphere of air for four hours at 550° C.

The Xray diffraction diagram of the crystals obtained from Example 4 shows the diffraction pattern typical for the MFI structure, and the IR spectrum the characteristic bands at 960 cm⁻¹. The UV/visible light spectrum shows that the sample is free from titanium dioxide and titanates.

Example 5 is performed analogously to Example 4, but with the use of the dispersion from Example 3.

In contrast to Example 4, Example 5 yields markedly more coarse zeolite aggregates. In the catalytic epoxidation of propylene, the product from Example 4 displays a higher activity than that from Example 5.

1: A dispersion comprising pyrogenic silicon-titanium mixed oxide powder with a silicon dioxide content of 75 to 99.99 wt % and a titanium dioxide content of 0.01 to 25 wt %,
water and
a basic, quaternary ammonium compound,
wherein the mean aggregate diameter of particles of the silicon-titanium mixed oxide powders in the dispersion is 200 nm at most.

2: The dispersion according to claim 1, wherein a mean aggregate diameter is less than 100 nm.

3: The dispersion according to claim 1, wherein a BET surface area of the pyrogenic silicon-titanium mixed oxide powder is 10 to 400 m²/g.

4: The dispersion according to claim 1, wherein said dispersion comprises at most 50 ppm of each of Na, K, Fe, Co, Ni, Al, Ca and Zn.

5: The dispersion according to claim 1, wherein the quaternary, basic ammonium compound is a tetralkylammonium hydroxide selected from the group consisting of tetraethylammonium hydroxide, tetra-n-propylammonium hydroxide, and tetra-n-butylammonium hydroxide.

6: The dispersion according to claim 1, wherein a mole water to mole silicon-titanium mixed oxide ratio ranges from 10 to 20.

7: The dispersion according to claim 1, wherein a pH value is 9 to 11.

8: The dispersion according to claim 1, wherein a mole ammonium compound to mole silicon-titanium mixed oxide ratio ranges from 0.12 to 0.20.

9: A process for the production of the dispersion according to claim comprising:
water, which, in the event that the silicon-titanium mixed oxide powder incorporated later leads to a pH value of the aqueous phase of <2 or >4, is adjusted to pH values of 2 to 4 by addition of acids or bases, is circulated from a holding tank through a rotor/ stator machine, and
via a filling device, silicon-titanium mixed oxide powder is continuously or discontinuously introduced into the shear zone between the grooves of the rotor teeth and the stator groove, with the rotor/stator machine running, in a quantity such that a predispersion with a solids content of 20 to 40 wt % results, and
after all the silicon-titanium mixed oxide powder has been added, the filling device is closed, and shearing continued such that the shear rate lies in the range between 10000 to 40000 sec⁻¹, and next, while maintaining the dispersing conditions, a basic, quaternary ammonium compound and optionally water, before the addition of the ammonium compound, are added.

10: A process for the production of a titanium-containing zeolite, comprising processing at a temperature of 150 to 220° .C for a time period of less than 12 hours the dispersion according to claim 1.

11: The process according to claim 10, further comprising separating, drying and calcinating the titanium-containing zeolite.

12: A titanium-containing zeolite obtained through the process according to claim 10.

13: A process for epoxidizing olefins comprising reacting an olefin with hydrogen peroxide in the presence of a titanium-containing zeolite according to claim 12, wherein said zeolite is a catalyst.

14: The process according to claim 10, wherein the dispersion further comprises a basic, quaternary ammonium compound.

15: A process for producing a dispersion according to claim 1, said process comprising:
(1) circulating water from a holding tank through a rotor/stator machine;
(2) introducing silicon-titanium mixed oxide powder into a shear zone, thereby forming a predispersion with a solids content of 20 to 40 wt %, where said shear zone is situated between grooves of rotor teeth and stator grooves;
(3) continually shearing said predispersion at a shear rate ranging from 10,000 to 40,000 sec⁻¹; and
(4) adding a basic, quaternary ammonium compound;
wherein, when the pH of said water in (1) is altered to less than 2 or greater than 4 by said introducing in (2), said pH is adjusted by adding an acid or a base.

16: The process according to claim 15, wherein (4) further comprises adding water.
